(19) United States
(12) Patent Application Publication
(10) Pub. No.: US 2008/0051793 A1
(43) Pub. Date: Feb. 28, 2008

(54) Drill-tap tool

(76) Inventors: David Erickson, Memphis, TN (US); Wilder Companioni, Cordova, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/499,190

(22) Filed: Aug. 4, 2006

(51) Int. Cl. A61B 17/58

(52) U.S. Cl. 606/73

(57) ABSTRACT

A tool having a drilling portion and a tapping portion may be used to form female threaded holes in bone. The tool may include a shaft having a drill tap portion on one end. In the drill tap portion, a tap section may be located adjacent to a drill section. The drill section may be configured to form an opening in bone while the tap section may be configured to form female threads in the walls of a hole in bone. The drill tap portion may further include a spiral flute to aid in the removal of debris. The tool may further include a stop portion configured to limit the depth of the tapped hole to a predetermined maximum depth that may correspond to the length of a particular fastener such as a bone screw. A handle attachment portion may be provided to facilitate the use of a drill.

The present disclosure relates to the field of bone instruments for drilling and tapping a hole in bone. In particular, this disclosure concerns a combined drill and tap that can be used with non-self-tapping or non-self-drilling bone screws, among other implants.

Some orthopedic procedures require the attachment of devices and/or plates to a bone. In particular, some orthopedic procedures performed on vertebral bodies may require the attachment of supports, plates and/or other devices to one or more vertebral bodies. Such devices may be attached to bone with bone screws. In some situations, it may be advantageous to use non-metallic screws, e.g. screws of a polymeric material such as PEEK or of resorbable materials. However, such non-metallic screws are often non-self drilling and non-self tapping. Insertion of such a screw may require the creation of a tapped hole in the bone or vertebral body. The creation of a tapped hole in a bone or vertebral body may require a pilot hole to be opened in the cortical bone. In the case of some vertebral bodies, the cortical bone may be three to five millimeters thick. After a pilot hole has been opened in the cortical bone, a tap could then be used to tap the pilot hole to create female threads to accept the insertion of the bone screw. This generally requires a multi-step process to complete.

A pilot hole in bone tissue could also be opened with a drill bit. A drill bit may include a shaft having a cutting section. One end of the shaft may be adapted to couple to a drill or other device that imparts rotational motion about a central longitudinal axis of the shaft. The opening may be formed in bone by rotating the drill bit and gradually moving the cutting section of the drill bit into the bone.

Alternatively, a pilot hole could be opened by puncturing the bone with a sharpened instrument such as an awl or trocar. The sharpened instrument may be forcefully inserted into the bone to form a pilot hole. Alternatively, or in addition, the sharpened instrument could be partially or completely rotated during insertion to assist in opening the pilot hole.

A tap may be used to form a thread flight in the previously opened pilot hole by inserting the tap in the hole and rotating the tap while driving it into the opening of the hole. The rotation of the tap forms a thread flight in the wall of the opening. One form of tap has male cutting edges that form a female thread in the wall of the opening. A tap may also include flutes that may define the male cutting edges and may provide channels for removing chips that are produced during the formation of the female thread flights.

In some orthopedic procedures that may require the insertion of a bone screw, it may be necessary to accurately and reliably control the depth of penetration of both the pilot hole forming step and the tapping step. For example, over drilling or over tapping a hole may damage some underlying soft tissue located beyond the desired screw location.

### SUMMARY OF THE DISCLOSURE

In view of the disadvantages in the known systems and methods used to drill and tap holes in bone, the present disclosure provides a new and useful combination drill-tap tool that may be used to accurately drill and tap female threaded holes in bone using a single step. This combination drill-tap tool may include a shaft having a drill-tap portion on one end. In the drill tap portion, a tap section may be located adjacent to a drill section. The drill section may be configured to form an opening in bone while the tap section may be configured to form female threads in the walls of a hole in bone. The drill tap portion may further include a spiral flute to aid in the removal of debris. The tool may further include a stop portion configured to limit the depth of the tapped hole to a predetermined maximum depth that may correspond to the length of a particular fastener such as a bone screw. A handle attachment portion may be provided to facilitate the use of a drill.

Aspects of this disclosure include a tool for making threaded surgical holes in bone that may include an elongated shaft having an operative portion, a hole cutting edge located on the operative portion and the edge being configured to bore a hole, a tap on the operative portion proximate to the hole cutting edge and being configured with a male thread form with multiple windings, so as to form female threads in bone, and a tap stop on the shaft proximate to and separate from the tap, the stop being configured to limit the maximum depth of the threaded surgical hole created by the tool to a predetermined depth. Such tools could also include a flute (e.g. a substantially helical flute) integrated with the tap and cutting across a plurality of the windings. An embodiment of the tap could have a front lead taper proximate to the hole cutting edge and a thread part which adjoins the front lead taper, with the thread part having teeth with a thread profile in cross section, and the front lead taper having a taper length as measured parallel to a longitudinal axis of said elongated shaft. Such a taper length can be shorter than a single thread pitch of the thread profile, and a slotted flute traversing a portion of said operative portion so as to intersect said thread part can be provided. In particular embodiments, a thread profile has a leading flank angle of about 10 degrees and a trailing flank angle of about 20 degrees, and/or the predetermined depth is not substantially greater than the length of a predetermined bone screw. The tap may be adjacent and operatively following the hole cutting edge, and the edge can be part of a trocar tip. A handle attachment can be located on the shaft and distal from the operative portion. The tap stop may be a portion of the shaft having a diameter substantially greater than a major diameter of the tap.

The disclosure also includes an orthopedic bone cutting tool for drilling and tapping a bore in bone, which can include a shaft having a tip, a shaft portion extending longitudinally from the tip having at least one male thread, and a non-threaded shaft portion extending longitudinally from the threaded shank portion. At least one cutting edge adapted and configured to cut a hole in bone for an orthopedic bone screw may extend through at least part of the threaded shaft portion, the at least one cutting edge causing the at least one male thread to be discontinuous. A flute beginning proximate to the tip and extending longitudinally into the threaded shaft portion can be provided, and the flute can extend completely through the threaded shaft portion. A stop portion located between the threaded shaft portion and at least part of the non-threaded shaft portion can also be provided, and the stop portion could have a non-threaded portion of the shaft between itself and the threaded shaft portion. In some embodiments, a transition portion is between the tip portion and the threaded shank portion, and has a transition thread with a reduced thread cross section. The tip may be a trocar tip, having a cutting edge for insertion into bone, as well as other types of cutting tips or surfaces.

Methods for using such tools, as to make a tapped hole in bone and/or insert a bone screw into it, can include one or more steps, such as providing a selection of different bone screws (e.g. non-metallic screws) having different screw lengths and possibly a thread cutting portion; selecting a particular bone screw having a desired screw length; providing a selection of different tools for making threaded surgical holes in bone able to create threaded holes of different predetermined maximum depths, wherein each of the different tools is specifically configured to create a hole with a maximum depth, wherein the maximum depth of the hole created is not significantly deeper than the screw length of the particular selected bone screw; selecting an appropriate tool configured to match the particular bone screw selected; substantially simultaneously boring and tapping a female threaded surgical hole to the maximum depth into bone determined by the selected tool using the selected tool; and/or inserting the particular bone screw into the female threaded surgical hole. To completely insert the particular bone screw into the female threaded surgical hole, a thread cutting portion of the particular bone screw may have to tap a thread proximate to the bottom of the threaded surgical hole.

### BRIEF DESCRIPTION OF THE DRAWINGS

FIG. 1 is a side elevational view of an embodiment of a combination drill and tap tool.

FIG. 2 is a partial side elevational view of an embodiment of a drill tap portion of the embodiment of FIG. 1.

FIG. 3 is a cross sectional representation of threads in the embodiment of the tap portion in the embodiment of FIG. 2.

FIG. 4 is an end view of a handle attachment portion of the embodiment of FIG. 1.

FIG. 5 is a partial side elevational view of another embodiment of a drill tap portion.

### DETAILED DESCRIPTION OF THE ILLUSTRATED EMBODIMENTS

For the purposes of promoting an understanding of the principles of the disclosure, reference will now be made to certain embodiments thereof and specific language will be used to describe the same. It will nevertheless be understood that no limitation of the scope of the claims is thereby intended, such alterations, further modifications and further applications of the principles of the disclosure as described herein being contemplated as would normally occur to one skilled in the art to which the disclosure relates.

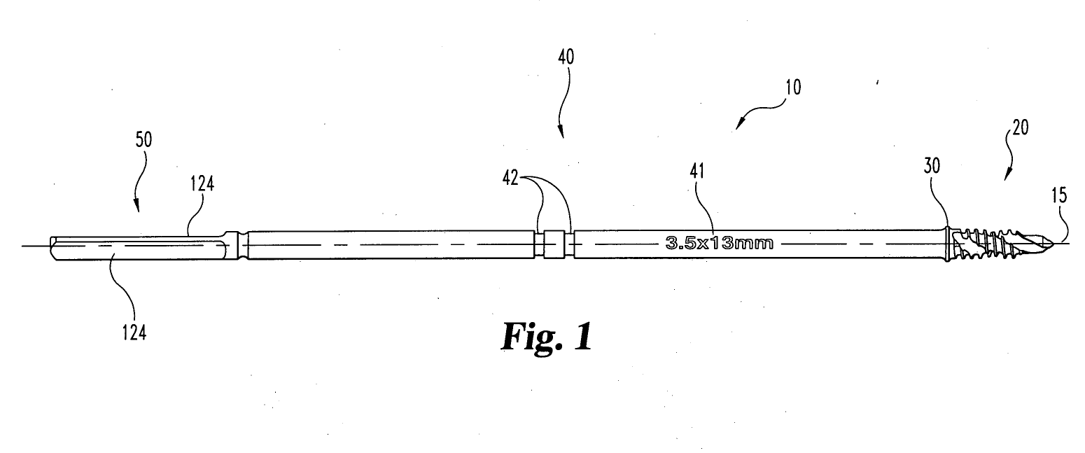

Referring to the figures, and particularly to FIG. 1, a combination drill and tap tool is designated generally as tool 10. Tool 10 may be used with other instruments such as a handle, drill or guide to form threaded openings in bone in a single operation. The threaded opening may extend through both cortical bone and cancellous bone. A thread flight formed by the tool 10 may be adapted to mate with a threaded fastener, such as a bone screw. Furthermore, the depth of the threaded opening may be adapted to be as deep, but no deeper than required, to allow the complete insertion of the threaded fastener into the threaded opening. Alternatively, the depth of the threaded opening may be adapted to require some further tapping of the threaded opening, possibly by the threaded fastener itself, to allow the complete insertion of the threaded fastener into the threaded opening.

Tool 10 may include a longitudinal axis 15, drill tap portion 20, stop portion 30, shaft portion 40 and handle attachment portion 50. Tool 10 may also include markings such as size etchings 41 along shaft portion 40 indicating the particular size of tool 10. Similarly, tool 10 may include color coded markings 42, such as a colored epoxy fill, located in the recesses found near the center of shaft portion 40 that could also indicate the particular size of tool 10. Tool 10 may be formed of any biocompatible material capable of drilling and tapping bone. One type of material that may be acceptable is medical grade stainless steel.

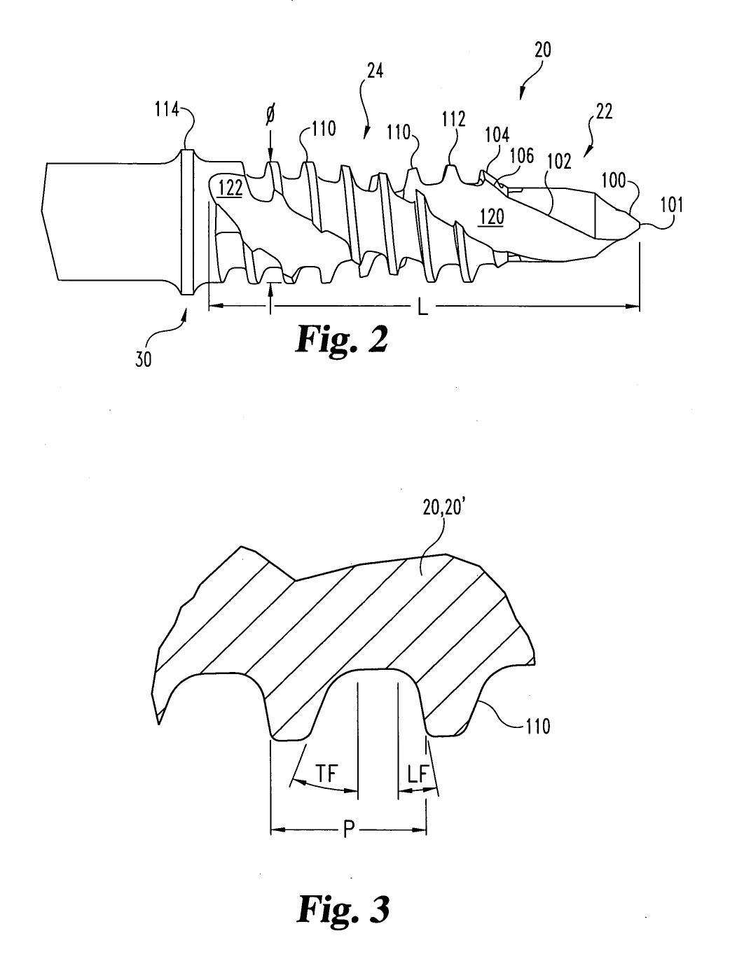

As shown in FIGS. 2 and 3, drill tap portion 20 may include drill portion 22 and tap portion 24. Tap portion 24 may overlap or be located adjacent to the drill portion 22. In an embodiment, tap portion 24 may abut the drill portion 22. Tap portion 24 may operatively follow drill portion 22, e.g. tap portion 24 may begin at a point that is closer to stop portion 30 than the point at which drill portion 22 begins. Drill portion 22 may include hole cutting edge 100, drill tip 101 and hole reaming edge 102, while tap portion 24 may include tap lead-in thread 104, lead-in cutting edge 106, full thread profile 110, thread cutting edge 112, major tap diameter Ø, spiral flute 120 and flute end 122.

Hole cutting edge 100 and drill tip 101 are generally configured to bore a hole in bone when tool 10 is rotated about longitudinal axis 15. Hole cutting edge 100, in combination with spiral flute 120 and hole reaming edge 102, may be configured similarly to a conventional drill bit for bone tissue, as is illustrated in one embodiment in FIGS. 1 to 3. In that embodiment, spiral flute 120 extends from hole cutting edge 100 along hole reaming edge 102 to remove bone or other debris cut by hole cutting edge 100 and hole reaming edge 102.

Drill tap portion 20, in this embodiment, includes a spiral flute 120. Tap portion 24 may be considered to be a male thread flight or thread part that has a plurality of cutting edges 112 separated and defined by spiral flute 120 such that the threaded part of drill tap portion 20 that includes thread profile 110 is made discontinuous by spiral flute 120. The cutting edges 112 of this embodiment generally form a female thread in bone. In one embodiment, drill tap portion 20 may have two spiral flutes 120 that are diametrically opposed or equally spaced around drill tap portion 20. Fewer, or more, than two flutes may be formed in drill tap portion 20 of tool 10 in specific embodiments. In addition, the flutes may be straight, instead of helically formed about drill tap portion 20 of tool 10. Spiral flute 120 may extend along the length of drill tap portion 20 to remove bone debris or other tissue or matter removed by cutting edges 106 and 112.

As already noted, lead-in cutting edge 106 and thread cutting edge 112 may be configured to tap a female threaded hole in bone when tool 10 is rotated about longitudinal axis 15. In the illustrated embodiment, edge 106 is formed at the junction of tap lead-in thread 104 and spiral flute 120. Similarly, thread cutting edge 112 is formed at the junctions of thread profile 114 and spiral flute 120. Tap lead-in thread 104 may have a thread profile that gradually introduces increasingly larger lead-in cutting edges 106 (e.g. having increasing crest width or crest diameter) and may reach a constant crest-diameter full thread profile 110. In this way, lead-in thread 104 and lead-in cutting edge 106 form a tapered transition portion in this embodiment between full thread profile 110 and drill portion 22, with lead-in thread 104 and lead-in cutting edge 106 having threads with comparatively reduced cross sections. In the embodiment illustrated in FIGS. 1 and 2, this gradual or tapered increase in lead-in thread 104 occurs in approximately one revolution about tool 10. In alternate embodiments, it is envisioned that this gradual or tapered increase in lead-in thread 104 may occur either more quickly (in fewer than one full revolution about tool 10) or more slowly (in more than one full revolution about tool 10). Similarly, the tapered transition portion of the illustrated embodiment is positioned proximate to drill tap portion 20 and adjoining full thread profile 110, which is the thread part of tap portion 24 in this embodiment.

FIG. 3 represents a partial cross-sectional view of an embodiment of full thread profile 110. Complete thread profile 110 may include leading flank angle LF, trailing flank angle TF and pitch P as illustrated in FIG. 3. Leading flank angle LF, trailing flank angle TF and pitch P may be configured to match the geometry of a specific threaded fastener such as a bone screw. In one embodiment, the leading flank angle could be about 10 degrees, the trailing flank angle could be about 20 degrees and pitch P could be about 1.2 mm.

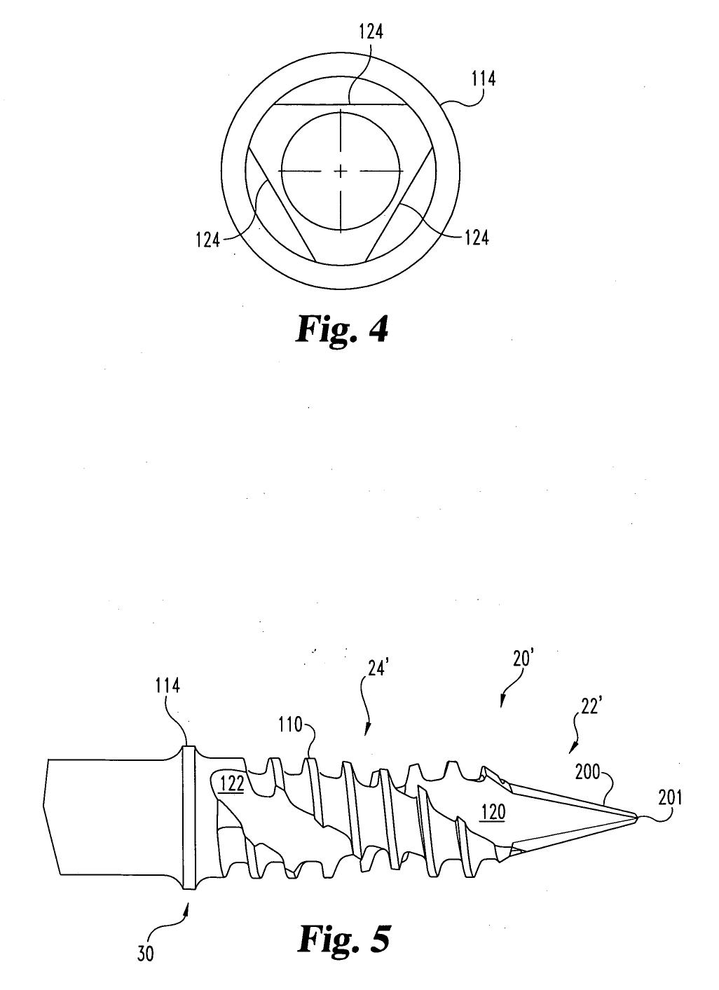

FIG. 4 is an end view of handle attachment portion 50. Handle attachment portion 50, as seen in the illustrated embodiment, has three flat surfaces 124 that make portion 50 an approximately equilateral-cross-section triangular solid, configured to interface with a handle or a chuck of a turning device such as a powered drill. Other types of attachment portion 50 could be used, such as those having single or double flat surfaces, hexagonal cross-section, or the like.

FIG. 5 illustrates another embodiment of a drill-tap portion 20′, which includes a trocar tip portion 22′ and tap portion 24′. Tap portion 24′ may overlap or be located adjacent to the trocar tip portion 22′. In an embodiment, tap portion 24′ may abut trocar tip portion 22′. Tap portion 24′ may operatively follow trocar tip portion 22′, e.g. tap portion 24′ may begin at a point that is closer to stop portion 30 than the point at which trocar tip portion 22′ begins. In the illustrated embodiment of portion 20′, trocar tip portion 22′ may include hole cutting edge 200 being configured to puncture and/or cut bone tissue, and its tip 201 intersects longitudinal axis 15. If configured to puncture, tip 201 can be sharpened to be insertable into cortical bone irrespective of rotation of tool 10. This may provide easier and more precise hole starting than drill tip 101. Trocar tip 22′ may be configured to cut bone either by motion along longitudinal axis 15 or by rotating portion 20′ about longitudinal axis 15. Spiral flute 120 may extend from hole cutting edge 200 to remove bone or other debris cut by hole cutting edge 200.

Tap portion 24′ may include tap lead-in thread 104, lead-in cutting edge 106, complete thread profile 110, thread cutting edge 112, major tap diameter Ø, spiral flute 120 and flute end 122, as previously disclosed. Lead-in thread 104 and lead-in cutting edge 106 may form a transition portion between the full thread profile 110 and drill portion 22′.

Stop portion 30 may include raised stop portion 114 located at a desired position along shaft portion 40 to limit the depth of any female threaded hole made by tool 10 to maximum depth L. Raised stop portion 114 may have an outer diameter that is substantially greater than the major tap diameter Ø. In one embodiment, the outer diameter of raised stop portion 114 may be 5 mm while the major tap diameter Ø is 4 mm. Alternatively, or in addition, flute end 122 of spiral flute 120 could limit the depth of any female threaded hole made by tool 10 to a predetermined maximum depth L. In various embodiments of tool 10, either raised stop portion 114 or flute end 122 may inhibit further drilling or tapping of a bone after the maximum depth L has been reached. Individual embodiments of tool 10 may be configured to specifically correspond to match the threaded length of individual screws such that individual embodiments of tool 10 may be specifically configured to drill and tap a female threaded hole to mate with a specific screw thread and screw length. Mating with a specific screw thread and screw length may entail drilling and tapping a hole that permits the complete insertion of the specific screw without any additional removal of bone from the hole. Alternatively, mating with a specific screw may entail drilling and tapping a hole that may require some additional thread tapping, possibly by the specific screw itself, possibly near the tip of the screw, to permit the complete insertion of the specific screw in the threaded hole in bone.

Configuring individual embodiments of tool 10 to specifically correspond to match the threaded length of individual screws may provide one or more of a variety benefits to the surgeon using tool 10. For example, having an individual tool 10 that specifically corresponds to an individual screw may provide holes with a more uniform depth than may be achievable by a similar tool that does not have a maximum depth L, or that can be used with a number of differently-configure screws. Furthermore, specifically tailoring tool 10 to an individual screw removes as a potential source of error in the surgeon's judgment in either setting an adjustable depth stop or a surgeon's judgment in determining a drilled and/or tapped hole is deep enough.

For example, in one embodiment of tool 10, maximum depth L could be equal to about 12.4 mm which may correspond to an individual screw having a length of 11 mm. In another embodiment, maximum depth L could be equal to 14.4 mm which may correspond to an individual screw having a length of 13 mm. Similarly, in yet another embodiment, maximum depth L could be equal to 16.4 mm which may correspond to an individual screw having a length of 15 mm. In yet another embodiment, maximum depth L could be equal to 18.4 mm which may correspond to an individual bone screw having a length of 17 mm. In this regard, the length of an individual bone screw is measured as the distance between the tip of the screw and the head of the screw.

In addition, specifically tailoring tool 10 to an individual screw allows the use of similar identification indicia on both tool 10 and the individual screw to further reduce any potential human error that could be encountered in the operating room wherein the incorrect tool could be selected for a particular screw. For example, it may be advantageous to mark the shaft portion 40 of tool 10 with a color coded size indicator. It may then be possible to mark some portion of the particular screw with a similar color coded size indicator that matches the size indicator utilized to mark tool 10. Color coding particular screws may also reduce misidentification of screws in the operating room.

Handle attachment portion 50 is attached to a drill or other appropriate turning tool, in certain embodiments, which can rotate tool 10 about longitudinal axis 15. Such an instrument may be a of a variety of types, including hand operated drills or a power drills, and may include features such as a slip clutch that inhibits application of excessive torque to tool 10 when tool 10 is forming a threaded hole in bone. Tool 10 is advanced while forming the threaded hole in bone at a rate generally related to the revolution speed of the tool and the pitch P of tap section 24 of tool 10. A hand operated drill may allow precise control of the revolution speed of the tool 10 to inhibit rapid advancement of the tool into bone.

In use, once tool 10 is connected to an appropriate drill, tool 10 may be rotated and pressed into bone so that drill portion 22 or trocar tip portion 22′ forms an opening in the bone. Tap portion 24 or tap portion 24′ may operatively follow into the opening formed by drill portion 22 or trocar tip portion 22′. Tap portion 24 or 24′ forms a thread in a wall of the opening as tool 10 is rotated and driven further into the bone. Thus, tool 10 performs both the functions of drilling a hole and tapping it in a single operation, and substantially simultaneously. Tool 10 may be advanced until stop portion 30 encounters bone tissue, or the edge of the hole drilled and tapped in it. At this point it may become increasingly difficult or impossible to rotate tool 10 about longitudinal axis 15, as stop portion 30 frictionally engages or binds against bone tissue. Tool 10 may be backed out of the threaded opening by reversing the direction of rotation used to form the threaded hole.

When initially positioning the location of a threaded hole in bone, trocar tip portion 22′ may advantageously allow the initial insertion of tool 10 into bone to occur exclusively with a driving or piercing force (using little or no rotation of tool 10). This may permit more accurate location of the threaded hole than may be possible utilizing drill portion 22. Furthermore, it may be possible to form an opening in the bone using trocar tip portion 22′ utilizing a rotating motion that alternates direction instead of utilizing unidirectional rotation. A rotating motion may advantageously allow the surgeon to keep their same hand position on a hand operated drill which may not be possible when using a hand drill for unidirectional rotation. This may permit more accurate formation of threaded holes in bone using a hand operated drill.

The devices disclosed herein are intended for uses related to orthopedic medicine. Accordingly, embodiments of tool 20, 20′ should be made of materials compatible with the environment of orthopedic surgery, such as stainless steel or other hard materials that can be effectively sterilized and can efficiently cut bone tissue. Cutting edges should be made so as to minimize or eliminate the chance of leaving rough edges in or on bone tissue or otherwise causing cuts, abrasions or punctures outside of the intended preparation of a hole in bone, or other tissue damage.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the claims are desired to be protected. Features described or shown with respect to one particular embodiment may be used or included with other embodiments consistent with this disclosure.

What is claimed is:

1. A tool for making threaded surgical holes in bone comprising:
an elongated shaft having an operative portion;
a hole cutting edge located on said operative portion, said edge being configured to bore a hole;
a tap on said operative portion proximate to said hole cutting edge, said tap being configured with a male thread form with multiple windings, so as to form female threads in bone;
a tap stop on said shaft proximate to and separate from said tap, said tap stop being configured to limit the maximum depth of the threaded surgical hole created by the tool to a predetermined depth.

2. The tool of claim 1, further comprising a flute integrated with said tap, said flute cutting across a plurality of said windings.

3. The tool of claim 2, wherein said flute is substantially helical.

4. The tool of claim 1, wherein said tap consists of a front lead taper proximate to the hole cutting edge and a thread part which adjoins said front lead taper, said thread part having teeth with a thread profile in cross section, said front lead taper having a taper length as measured parallel to a longitudinal axis of said elongated shaft.

5. The tool of claim 4, wherein said taper length is shorter than a single thread pitch of said thread profile.

6. The tool of claim 4, further comprising a slotted flute traversing a portion of said operative portion so as to intersect said thread part.

7. The tool of claim 4, wherein said thread profile has a leading flank angle of about 10 degrees and a trailing flank angle of about 20 degrees.

8. The tool of claim 1, wherein said predetermined depth is not substantially greater than the length of a predetermined bone screw.

9. The tool of claim 1, wherein said tap is adjacent and operatively follows said hole cutting edge.

10. The tool of claim 1, wherein said hole cutting edge is part of a trocar tip.

11. The tool of claim 1, further comprising a handle attachment located on said shaft, said handle attachment being distal from said operative portion.

12. The tool of claim 1, wherein said tap stop comprises a portion of said shaft having a diameter substantially greater than a major diameter of said tap.

13. An orthopedic bone cutting tool for drilling and tapping a bore in bone comprising:
a shaft having a tip, a shaft portion extending longitudinally from said tip having at least one male thread, and a non-threaded shaft portion extending longitudinally from said threaded shank portion;
at least one cutting edge adapted and configured to cut a hole in bone for an orthopedic bone screw, said at least one cutting edge extending through at least part of said threaded shaft portion, said at least one cutting edge causing said at least one male thread to be discontinuous.

14. The device of claim 13, further comprising a flute beginning proximate to said tip and extending longitudinally into said threaded shaft portion.

15. The device of claim 14, wherein said flute extends completely through said threaded shaft portion.

16. The device of claim 13, further comprising a stop portion located between said threaded shaft portion and at least part of said non-threaded shaft portion.

17. The device of claim 16, wherein said stop portion has a non-threaded portion of said shaft between itself and said threaded shaft portion.

18. The device of claim 13, further comprising a transition portion between said tip portion and said threaded shank portion, said transition portion having a transition thread having a reduced thread cross section.

19. The device of claim 13, wherein said tip is a trocar tip, having a cutting edge for insertion into bone.

20. A method of inserting a bone screw into bone comprising:
providing a selection of different bone screws having different screw lengths;
selecting a particular bone screw having a screw length;
providing a selection of different tools for making threaded surgical holes in bone able to create threaded holes of different predetermined maximum depths, wherein each of the different tools is specifically configured to create a hole with a maximum depth, wherein the maximum depth of the hole created is not significantly deeper than the screw length of the particular bone screw;
selecting the appropriate tool configured to match the particular bone screw selected;
substantially simultaneously boring and tapping a female threaded surgical hole to the maximum depth into bone determined by the selected tool using the selected tool;
inserting the particular bone screw into the female threaded surgical hole.
